(19) United States
(12) Patent Application Publication
(10) Pub. No.: US 2012/0295833 A1
(43) Pub. Date: Nov. 22, 2012

(54) Polysaccharides having an adjustable degree of functionalization

(75) Inventors: Richard CHARVET, Rillieux La Pape (FR); Guilhem MORA, Lyon (FR); Rémi SOULA, Lyon (FR)

(73) Assignee: ADOCIA, Lyon (FR)

(21) Appl. No.: 13/468,799

(22) Filed: May 10, 2012

Related U.S. Application Data
(60) Provisional application No. 61/541,606, filed on Sep. 30, 2011.
(60) Provisional application No. 61/484,461, filed on May 10, 2011.

(30) Foreign Application Priority Data
May 10, 2011 (FR) 1154039
Sep. 30, 2011 (FR) 1158885

(51) Int. Cl. C08B 37/02; A61K 38/02; A61K 47/36

(52) U.S. Cl. 514/1.1; 536/51; 514/777

(57) ABSTRACT

An anionic polysaccharide comprising carboxylic acid functional groups of formula I:

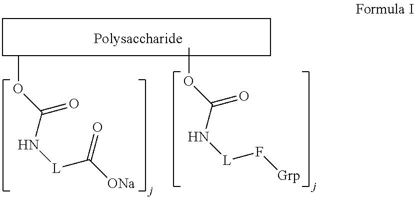

in which: the polysaccharide may be either, synthetic or modified, i represents the degree of functionalization of the hydroxyls, j represents the degree of functionalization of the hydroxyls, L and Q represent an optionally branched and/or unsaturated chain comprising from 1 to 15 carbons, F is a functional group chosen from the group consisting of the carbamate functional group, the carbonyl being bonded to the Q radical, the ester functional group, the carbonyl being bonded to the Q radical, the amide functional group, the carbonyl being bonded to the Q radical, and the carboxylate functional group, and Grp is chosen from the group consisting of a mono- or divalent cation and of a chain comprising from 4 to 50 carbons.

The present invention relates to anionic polysaccharides intended for a therapeutic and/or prophylactic use in the administration of active principle(s) to man or animals.

Polysaccharides comprising carboxyl groups are, due to their structure and their biocompatibility, of a degree of interest to the pharmaceutical industry, in particular for the stabilization of active principles, for example protein active principles, by the formation of complexes.

The functionalization of these polysaccharides by carboxylate groups or carboxylic acid functional groups advantageously makes it possible to adjust the interaction forces involved between the polysaccharide and the active principle in order to promote the formation of complexes.

For the requirements of the present patent application, and as indicated below in the description and claims, the term “functionalization by carboxylate groups” or “carboxylic acid functional groups” is understood to mean the necessary presence of at least one carboxylate group or carboxylic acid functional group in at least one of the substituents of the hydroxyl group of the polysaccharide.

In this context, WO 2008/038111 describes dextrans modified by chains of the general formula:

—R—[AA]

in which:
the free end [AA] denotes a hydrophobic amino acid residue (for example, a tryptophan residue) attached to the dextran backbone via a connecting arm R,
the connecting arm R is a C₁ to C₁₈ carbon chain which is optionally substituted and which comprises at least one heteroatom (such as O, N and S) and at least one acid functional group; it forms an amide bond with the amino acid residue [AA] and is directly attached to the dextran backbone via a bond of ester, thioester, amide, carbonate, carbamate, ether, thioether or amine type, preferably an ether bond.

Applied to the delivery of active principles, such as insulin and PDGF-BB, these modified dextrans have already proved their effectiveness. However, the inventors have established that, for other active principles, variations with regard to the structure of the modified dextran were necessary, in particular the density of anionic charges of the modified dextran. In point of fact, the modified dextrans described, due to their structure and method of synthesis, offer a limited ability to be adjusted, with a maximum functionalization of two carboxylate groups per glycoside unit, which restricts their field of application and their potential for development as pharmaceutical excipients.

Furthermore, from the viewpoint of the therapeutic application targeted, the degradability of the polymers is a determining property. Surprisingly, the inventors have been able to demonstrate that the polymers comprising urethane functional groups on the polysaccharide backbone exhibit a better compromise in terms of stability under the storage conditions and of degradability after administration.

The literature reports several examples of the grafting of isocyanate to polysaccharides. Dextran in particular has been functionalized by butyl isocyanate (Arranz, F. et al., Makromol. Chem., 1987, 188, 2831-2838) or an isocyanate derivative of polyethylene glycol (Won, C-Y., Polymer Bulletin, 2004, 52, 109-115). These modified dextrans are neutral and thus do not carry any carboxylate group. In the case of the dextran modified by butyl chains, the solubility in water is greatly reduced, the objective pursued being the formation of hydrophobic dextran derivatives. In the case of the dextran modified by chains of polyethylene glycol type, the polymers obtained cannot interact with proteins in so far as polyethylene glycol is known to be furtive with regard to proteins. Furthermore, these polyethylene glycol chains carry terminal acrylate functional groups in order to crosslink them in order to form hydrogels. No possibility of the formation of a complex with a therapeutic molecule can be envisaged for these two types of polymer.

The present invention is targeted at providing polysaccharides, intended for the stabilization, administration and delivery of active principles, which can be prepared by methods which are relatively simple to employ and which offer an increased ability to be adjusted in terms of interaction properties. The aim of the present invention is thus to provide polysaccharides capable of making possible the stabilization, administration and delivery of a great diversity of active principles.

The present invention is also targeted at obtaining polysaccharides with a degree of functionalization which can range beyond two carboxylate groups per saccharide unit.

The present invention is also targeted at providing polysaccharides which are biodegradable in vivo, making it possible to obtain, after hydrolysis of the functional groups, the native polysaccharide, the safety and removal of which are documented. The invention is also targeted at producing polysaccharides which can exhibit a biodegradability which is sufficiently fast and appropriate for the use thereof in the preparation of a broad category of pharmaceutical formulations, including for medicaments intended for a chronic and/or high frequency administration. Apart from the requirement of a biodegradability which can be adjusted after administration, the invention is targeted at providing polysaccharides which meet the constraints drawn up by the pharmaceutical industry, in particular in terms of stability under normal conditions of preservation and storage and in particular in solution.

The present invention relates to an anionic polysaccharide chosen from polysaccharides comprising carboxylic acid functional groups of formula I:

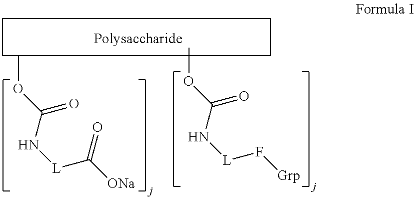

in which:
the said polysaccharide is chosen from the group consisting of dextran, alginate, hyaluronan and galacturonan, these being natural, synthetic or modified,
i represents the degree of functionalization of the hydroxyls by L-CO₂Na per saccharide unit and 0<i≦3, and
j represents the degree of functionalization of the hydroxyls as -Q-F-Grp per saccharide unit and 0≦j≦1.5 with 0<i+j≦3, and
if j=0, then L is not —CH₂—,
L and Q, which are different, represent an optionally branched and/or unsaturated chain comprising from 1 to 15 carbons, optionally comprising one or more heteroatoms, such as O, N and/or S, and optionally comprising one or more saturated, unsaturated or aromatic rings or heterocycles, and are chosen from the radicals of formulae VII and VIII:

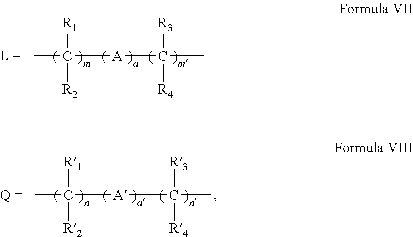

in which:
m and n, which are identical or different, are greater than or equal to 1 and less than or equal to 12,
m′ and n′, which are identical or different, are less than or equal to 12,
A and A′, which are identical or different, are functional groups chosen from the group consisting of the amide functional group, the carbonyl being bonded to the —Cₘ(R₁R₂)— radical, such as —C(R₁R₂)—CO—NH—C(R₃R₄)—, or bonded to the —Cₙ(R′₁R′₂)— radical, such as —C(R′₁R′₂)—CO—NH—C(R′₃R′₄)—, and the carbamate functional group,
R₁ and R₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₁ and R′₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R₃ and R₄, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₃ and R′₄, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
a and a′, which are identical or different, are equal to 0 or 1,
when R₁ and R₂ are hydrogen atoms and when a=0 and m′=0, then m is other than 1,
when a′=1, then L is not than:

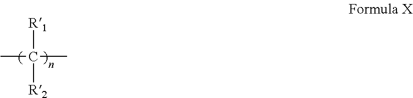

in which R′₁, R′₂ and n have the values defined above,
F is a functional group chosen from the group consisting of the carbamate functional group, the ester functional group, the carbonyl being bonded to the Q radical, such as -Q-CO—O-Grp, the amide functional group, the carbonyl being bonded to the Q radical, such as -Q-CO—NH-Grp, and the carboxylate functional group, the carbonyl being bonded to the Q radical, such as -Q-CO—OH,
Grp is chosen from the group consisting of a mono- or divalent cation, if the —NH-Q-F— radical results from an amino acid, F being a carboxylate functional group, and of a chain comprising from 4 to 50 carbons, which is optionally branched and/or unsaturated, and/or comprising one or more heteroatoms, such as O, N and/or S, in the form of non-terminal functional groups, and/or comprising one or more saturated, unsaturated or aromatic rings or heterocycles, and
the carboxylic acid functional groups are in the form of carboxylates of mono- or divalent cations.

The cations can be chosen from alkali metal cations, such as Na⁺ or K⁺.

In the definition of Grp, the term “non-terminal functional groups” is understood to mean functional groups chosen from the group consisting of ester, amide, carbamate or secondary or tertiary amine functional groups, carboxylate functional groups being excluded.

In one embodiment, the anionic polysaccharide according to the invention is characterized in that it is chosen from polysaccharides of formula I in which j is other than 0.

In one embodiment, j=0.

In one embodiment, 0≦j≦1 with 0<i+j≦3.

In one embodiment, a=0.

In one embodiment, a′=0.

In one embodiment, m′=0.

In one embodiment, n′=0.

In one embodiment, the anionic polysaccharide according to the invention is characterized in that it is chosen from the polysaccharides of formula I, in which:
i, j, L and Q have the meanings given above,
F is a functional group chosen from the group consisting of the carbamate functional group, the ester functional group, the carbonyl being bonded to the Q radical, such as -Q-CO—O-Grp, and the amide functional group, the carbonyl being bonded to the Q radical, such as -Q-CO—NH-Grp,
Grp is chosen from the group consisting of a chain comprising from 4 to 50 carbons, which is optionally branched and/or unsaturated, and/or comprising one or more heteroatoms, such as O, N and/or S, in the form of non-terminal functional groups, and/or comprising one or more saturated, unsaturated or aromatic rings or heterocycles,
the carboxylic acid functional groups are in the form of carboxylates of mono- or divalent cations.

In one embodiment, the anionic polysaccharide according to the invention is characterized in that it is chosen from the polysaccharides of formula I, in which:
i, j, F, L and Q have the meanings given above,
Grp is chosen from the group consisting of a mono- or divalent cation, if the —NH-Q-F— radical results from an amino acid, F being a carboxylate functional group, a saturated or unsaturated and linear, branched or cyclic C₄ to C₁₆ alkyl, a benzyl, an alkylaryl and a cholesteryl,
the carboxylic acid functional groups are in the form of carboxylates of mono- or divalent cations.

In one embodiment, the anionic polysaccharide according to the invention is characterized in that it is chosen from the polysaccharides of formula IX:

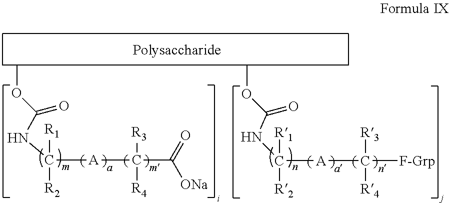

in which:
i, j, m, m′, a, a′, n and n′ have the meanings given above,
A and A′, which are identical or different, are functional groups chosen from the group consisting of the amide functional group, the carbonyl being bonded to the —Cₘ(R₁R₂)— radical, such as —C(R₁R₂)—CO—NH—C(R₃R₄)—, or bonded to the —Cₙ(R′₁R′₂)— radical, such as —C(R′₁R′₂)—CO—NH—C(R′₃R′₄)—, and the carbamate functional group,
R₁ and R₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₁ and R′₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R₃ and R₄, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₃ and R′₄, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
F is a functional group chosen from the group consisting of the carbamate functional group, the ester functional group, the carbonyl being bonded to the Q radical, such as -Q-CO—O-Grp, the amide functional group, the carbonyl being bonded to the Q radical, such as -Q-CO—NH-Grp, and the carboxylate functional group, the carbonyl being bonded to the Q radical, such as -Q-CO—OH,
Grp is chosen from the group consisting of a mono- or divalent cation, if the —NH-Q-F-radical results from an amino acid, F being a carboxylate functional group, a saturated or unsaturated and linear, branched or cyclic C₁ to C₆ alkyl, a benzyl, an alkylaryl and a cholesteryl, and
the carboxylic acid functional groups are in the form of carboxylates of mono- or divalent cations.

In one embodiment, the anionic polysaccharide according to the invention is characterized in that it is chosen from the polysaccharides of formula I, in which:
j, F and Grp have the meanings given above,
a=0, a′=0, m′=0, n′=0,
L and Q, which are different, are chosen from the radicals of formulae II and III:

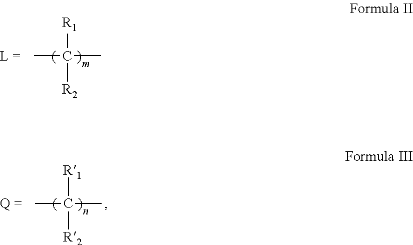

in which:
m and n, which are identical or different, are greater than or equal to 1 and less than or equal to 12, and
if j=0, then m is not 1 when R₁ and R₂ are hydrogen atoms,
R₁ and R₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₁ and R′₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH phenol functional groups.

In one embodiment, the anionic polysaccharide according to the invention is characterized in that it is chosen from the polysaccharides of formula IX, in which:
i, j, L and Q have the meanings given above, and
F is a functional group chosen from the group consisting of the carbamate functional group, the ester functional group, the carbonyl being bonded to the Q radical, such as -Q-CO—O-Grp, and the amide functional group, the carbonyl being bonded to the Q radical, such as -Q-CO—NH-Grp,
Grp is chosen from the group consisting of a chain comprising from 4 to 50 carbons, which is optionally branched and/or unsaturated, and/or comprising one or more heteroatoms, such as O, N and/or S, in the form of non-terminal functional groups, and/or comprising one or more saturated, unsaturated or aromatic rings or heterocycles.

In one embodiment, the anionic polysaccharide according to the invention is characterized in that it is chosen from the polysaccharides of formula IV:

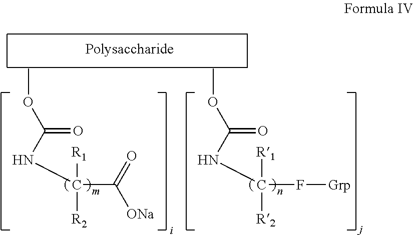

in which:
i, j, m and n have the meanings given above,
R₁ and R₂, which are identical or different, are chosen from the group consisting of a hydrogen atom, a saturated or unsaturated and linear, branched or cyclic C₁ to C₆ alkyl, a benzyl and an alkylaryl, the said groups optionally comprising carboxylic acid functional groups,
R′₁ and R′₂, which are identical or different, are chosen from the group consisting of a hydrogen atom, a saturated or unsaturated and linear, branched or cyclic C₁ to C₆ alkyl, a benzyl and an alkylaryl,
F is a functional group chosen from the group consisting of the carbamate functional group, the ester functional group, the carbonyl being bonded to the Q radical, such as -Q-CO—O-Grp, the amide functional group, the carbonyl being bonded to the Q radical, such as -Q-CO—NH-Grp, and the carboxylate functional group, the carbonyl being bonded to the Q radical, such as -Q-CO—OH,
Grp is chosen from the group consisting of a mono- or divalent cation, if the —NH-Q-F-radical results from an amino acid and F is a carboxylate functional group, a saturated or unsaturated and linear, branched or cyclic C₁ to C₆ alkyl, a benzyl, an alkylaryl and a cholesteryl, and
the carboxylic acid functional groups are in the form of a salt.

In one embodiment, the polysaccharide is characterized in that the L radical is chosen from the group consisting of the radicals of formula —(CH₂)ₒ— with 1≦o≦6.

In one embodiment, the polysaccharide is characterized in that the L radical is the —(CH₂)— radical.

In one embodiment, the polysaccharide is characterized in that the L radical is chosen from the group consisting of the following radicals:

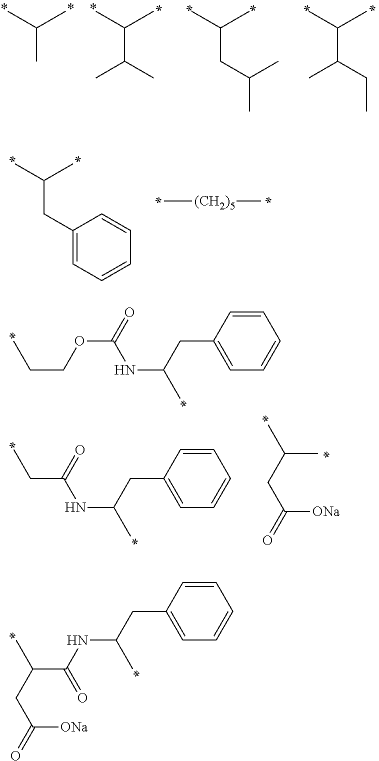

In one embodiment, the polysaccharide is characterized in that the Q radical is chosen from the group consisting of the following radicals:

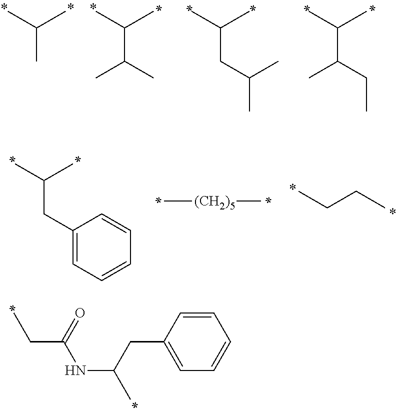

In one embodiment, the polysaccharide is characterized in that the —NH-Q-radical results from a diamine.

In one embodiment, the diamine is chosen from the group consisting of ethylenediamine and lysine and its derivatives.

In one embodiment, the diamine is chosen from the group consisting of diethylene glycol diamine and triethylene glycol diamine.

In one embodiment, the polysaccharide is characterized in that the —NH-Q-radical results from an alkanolamine.

In one embodiment, the alkanolamine is chosen from the group consisting of ethanolamine, diethylene glycol amine and triethylene glycol amine.

In one embodiment, the polysaccharide is characterized in that the —NH-Q-radical or the —NH-L-CO₂Na— radical results from an amino acid.

In one embodiment, the amino acids are chosen from α-amino acids.

In one embodiment, the α-amino acids are chosen from natural α-amino acids.

In one embodiment, the natural α-amino acids are chosen from leucine, alanine, isoleucine, glycine, phenylalanine and valine.

In one embodiment, the polysaccharide is characterized in that the —NH-L-CO₂Na— radical results from an amino acid chosen from the group consisting of aspartic acid and glutamic acid.

In one embodiment, the polysaccharide is characterized in that the Grp radical is chosen from the group consisting of the radicals of formula:

—[CH₂]ₚ—CH₃ with 4≦p≦12.

In one embodiment, the polysaccharide is characterized in that the Grp radical is a benzyl radical.

In one embodiment, the polysaccharide is characterized in that the Grp radical results from a hydrophobic alcohol.

In one embodiment, the hydrophobic alcohol is chosen from the alcohols composed of a saturated or unsaturated and/or branched or unbranched alkyl chain comprising from 4 to 18 carbons.

In one embodiment, the hydrophobic alcohol is chosen from the alcohols composed of a saturated or unsaturated and/or branched or unbranched alkyl chain comprising 6 to 18 carbons.

In one embodiment, the hydrophobic alcohol is chosen from the alcohols composed of a saturated or unsaturated and/or branched or unbranched alkyl chain comprising 8 to 16 carbons.

In one embodiment, the hydrophobic alcohol is octanol.

In one embodiment, the hydrophobic alcohol is 2-ethylbutanol.

In one embodiment, the fatty alcohol is chosen from myristyl alcohol, cetyl alcohol, stearyl alcohol, cetearyl alcohol, butyl alcohol, oleyl alcohol and lanolin.

In one embodiment, the hydrophobic alcohol is chosen from the group consisting of cholesterol and its derivatives.

In one embodiment, the hydrophobic alcohol is cholesterol.

In one embodiment, the hydrophobic alcohol is chosen from menthol derivatives.

In one embodiment, the hydrophobic alcohol is menthol in its racemic form.

In one embodiment, the hydrophobic alcohol is the D isomer of menthol.

In one embodiment, the hydrophobic alcohol is the L isomer of menthol.

In one embodiment, the hydrophobic alcohol is chosen from tocopherols.

In one embodiment, the tocopherol is α-tocopherol.

In one embodiment, the α-tocopherol is the racemate of α-tocopherol.

In one embodiment, the tocopherol is the D isomer of α-tocopherol.

In one embodiment, the tocopherol is the L isomer of α-tocopherol.

In one embodiment, the hydrophobic alcohol is chosen from alcohols carrying an aryl group.

In one embodiment, the alcohol carrying an aryl group is chosen from the group consisting of benzyl alcohol and phenethyl alcohol.

In one embodiment, 0.3≦i≦2.6.

In one embodiment, 0.4≦i≦2.2.

In one embodiment, 0.5≦i≦1.8.

In one embodiment, 0.6≦i≦1.4.

In one embodiment, 0.05≦j≦0.8.

In one embodiment, 0.1≦j≦0.7.

In one embodiment, 0.15≦j≦0.6.

In one embodiment, 0.2≦j≦0.5.

In one embodiment, 0.05≦j≦1.5.

In one embodiment, 0.1≦j≦0.8.

In one embodiment, 0.15≦j≦0.7.

In one embodiment, 0.2≦j≦0.6.

In one embodiment, 0.3≦j≦0.5.

The polysaccharide according to the invention has a degree of polymerization of between 3 and 10 000.

In one embodiment, it has a degree of polymerization of between 10 and 1000.

In another embodiment, it has a degree of polymerization of between 15 and 500.

The polysaccharides having a degree of polymerization of less than 10 are commonly referred to as oligosaccharides.

Anionic is understood to mean a polysaccharide which comprises non-functionalized and salifiable carboxylic acid functional groups.

Degree of functionalization of hydroxyl functional groups is understood to mean the mean number of hydroxyls functionalized in the carbamate form per saccharide unit.

Degree of polymerization DP is understood to mean the mean number of repeat units (monomers) per polymer chain. It is calculated by dividing the number-average molar mass by the average molar mass of the repeated unit.

Number-average molar mass (Mₙ) is understood to mean the arithmetic mean of the masses of each of the polymer chains. Thus, for a number nᵢ of chains i of molar mass Mᵢ, Mₙ=(ΣᵢnᵢMᵢ)/(Σᵢnᵢ).

The weight-average molar mass (M_{w}) is obtained by M_{w}=(ΣᵢnᵢMᵢ²)/(ΣᵢnᵢMᵢ), nᵢ being the number of polymer chains i of molar mass Mᵢ.

The polymers can also be characterized by the distribution of chain lengths, also referred to as polydispersity index (PI), which is equal to M_{w} divided by Mₙ.

In the definition of F, when F is a functional group chosen from the group consisting of the ester and amide functional groups, the expression “the carbonyl bonded to the Q radical” is understood to mean the -Q-F-Grp sequences represented below:

-Q-CO—O-Grp, -Q-CO—NH-Grp

In the representations, for example of L and Q, the asterisks * represent the pendent bond which will allow the Q radical represented in the formula I to be bonded to F or to the nitrogen of the carbamate functional group and which will allow the L radical represented in the formula I to be bonded to the carboxylate functional group or to the nitrogen of the carbamate functional group.

In one embodiment, the polysaccharide is a natural polysaccharide and is chosen from the group consisting of dextran, alginate, hyaluronan and galacturonan.

In one embodiment, the polysaccharide is a polysaccharide naturally carrying carboxylic acid functional groups and is chosen from the group consisting of alginate, hyaluronan and galacturonan.

In one embodiment, the polysaccharide is a naturally neutral polysaccharide and is dextran.

In one embodiment, the polysaccharide is chosen from the group consisting of alginate and hyaluronan.

In one embodiment, the polysaccharide is an alginate.

In one embodiment, the polysaccharide is a hyaluronan.

In one embodiment, the polysaccharide is a modified synthetic polysaccharide obtained from a polysaccharide chosen from the group consisting of dextran, alginate, hyaluronan and galacturonan substituted, by etherification of at least one hydroxyl functional group of the polysaccharide, by a radical carrying a carboxylic acid functional group, the said radical being chosen from the group consisting of the radicals of following formula V:

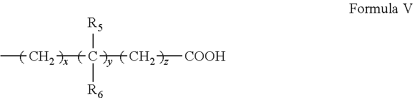

in which:
1≦x+y+z≦6, 0≦x≦3, 0≦y≦3 and 0≦z≦3
R₅ and R₆, which are identical or different, are chosen from the group consisting of —H, linear or branched C₁ to C₃ alkyl, —COON and the radical of general formula VI:

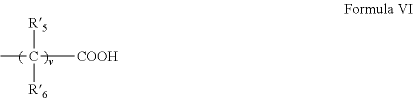

in which:
1≦v≦3 and
R′₅ and R′₆, which are identical or different, are chosen from the group consisting of —H and a linear or branched C₁ to C₃ alkyl group.

In one embodiment, x+y+z≦5.

In one embodiment, x+y+z≦4.

In one embodiment, the synthetic modified polysaccharide is a carboxymethyldextran.

In one embodiment, the invention relates to a polysaccharide chosen from the group consisting of the following polysaccharides:
dextran modified by sodium L-phenylalaninate carbamate,
dextran modified by sodium N-methylcarboxylate carbamate and sodium L-phenylalaninate carbamate,
dextran modified by sodium N-methylcarboxylate carbamate and octyl phenylalaninate carbamate,
dextran modified by sodium L-aspartate acid carbamate and sodium L-phenylalaninate carbamate,
dextran modified by sodium 6-hexanoate carbamate and benzyl phenylalaninate carbamate,
dextran modified by sodium 6-hexanoate carbamate and sodium L-leucinate carbamate,
dextran modified by sodium L-phenylalaninate L-aspartate carbamate,
dextran modified by sodium 2-[(2-aminoethoxycarbonyl)amino]-3-phenylpropanoate carbamate,
dextran modified by sodium N-methylcarboxylate carbamate and cholesteryl leucinate carbamate,
dextran modified by sodium N-methylcarboxylate carbamate and cholesteryl 1-ethylenediamine carboxylate carbamate.

The invention also relates to the synthesis of the polysaccharides according to the invention.

In one embodiment, a stage of functionalization of the hydroxyls of the polysaccharide is carried out by grafting a CH₃O₂C-L-N═C═O or CH₃CH₂O₂C-L-N═C═O compound, a compound carrying an isocyanate functional group and a methyl ester or ethyl ester functional group. During the purification stage, the carboxylate charges are formed by basic hydrolysis of the ester functional groups carried by the grafted compound.

In one embodiment, the functionalized polysaccharide is obtained by grafting a CH₃O₂C-L-N═C═O or CH₃CH₂O₂C-L-N═C═O compound and a Grp-F-Q-N═C═O compound (F not being an ester functional group), each compound carrying a reactive isocyanate functional group. During the purification stage, the carboxylate charges are formed by basic hydrolysis of the methyl or ethyl ester functional groups present on one of the grafted compounds.

In one embodiment, the functionalized polysaccharide is obtained by grafting a CH₃O₂C-L-N═C═O or CH₃CH₂O₂C-L-N═C═O compound. During the purification stage, the carboxylate charges are formed by basic hydrolysis of the methyl or ethyl ester functional groups present on one of the grafted compounds. The anionic polymer obtained is then acidified, lyophilized and then functionalized by grafting a Grp-F-Q-N═C═O compound.

In one embodiment, the isocyanate compounds are grafted by basic catalysis, as indicated in the publication Arranz, F. et al., Makromol. Chemie, 1987, 188, 2831-2838.

In one embodiment, the isocyanate compounds are grafted by catalysis using a tin derivative, as indicated in the publication Engelmann, F. et al., Starke, 2001, 53, 109-115.

In one embodiment, the isocyanate compounds are grafted by heating, as indicated in the publication Shen, X. et al., Polymer Bulletin, 2005, 55, 317-322.

In one embodiment, the preparation of the polysaccharides of formula I comprises a stage of obtaining an isocyanate intermediate CH₃O₂C-L-N═C═O or CH₃CH₂O₂C-L-N═C═O and an isocyanate intermediate Grp-F-Q-N═C═O.

In one embodiment, the isocyanate intermediate Grp-F-Q-N═C═O is obtained from an amine intermediate Grp-F-Q-NH₂ and/or ammonium salt Grp-F-Q-NH₃⁺, the counterion of which is an anion chosen from halides, sulphates, sulphonates and carboxylates.

The invention also relates to the use of the functionalized polysaccharides according to the invention in the preparation of pharmaceutical compositions.

The invention also relates to a pharmaceutical composition comprising one of the polysaccharides according to the invention as described above and at least one active principle.

The invention also relates to a pharmaceutical composition, characterized in that the active principle is chosen from the group consisting of proteins, glycoproteins, peptides and non-peptide therapeutic molecules.

Active principle is understood to mean a product in the form of a single chemical entity and/or in the form of a combination having a physiological activity. The said active principle can be exogenous, that is to say that it is introduced by the composition according to the invention. It can also be endogenous, for example the growth factors which will be secreted in a wound during the first phase of healing and which may be retained on the said wound by the composition according to the invention.

According to the pathologies targeted, it is intended for a local and/or systemic treatment.

In the case of local and systemic releases, the methods of administration envisaged are by the following routes: intravenous, subcutaneous, intradermal, transdermal, intramuscular, oral, nasal, vaginal, ocular, buccal, pulmonary, and the like.

The pharmaceutical compositions according to the invention are either in the liquid form, in aqueous solution, or in the powder, implant or film form. They additionally comprise the conventional pharmaceutical excipients well known to a person skilled in the art.

Depending on the pathologies and the methods of administration, the pharmaceutical compositions may advantageously comprise, in addition, excipients which make it possible to formulate them in the form of a gel, sponge, injectable solution, solution to be taken orally, Lyoc, and the like.

The invention also relates to a pharmaceutical composition, characterized in that it can be administered in the form of a stent, of a film or coating of implantable biomaterials, or of an implant.

### EXAMPLES

Examples of polymers which can be used in the compositions according to the invention are presented, without implied limitation, in Table 1 below.

TABLE 1

| Examples | —O—C(O)—NH—L—C(O)O—Na | —O—C(O)—NH—Q—F-Grp |
|---|---|---|
| Polymer 1 Dextran modified by sodium L-phenylalaninate carbamate DP = 19 i = 0.9 j = 0 Polymer 11 Dextran modified by sodium L-phenylalaninate carbamate DP = 19 i = 2 j = 0 Oligomer 14 Dextran modified by sodium L-phenylalaninate carbamate DP = 4 i = 2.1 j = 0 | 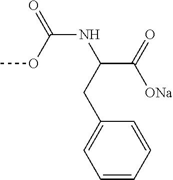 | N/A |
| Polymer 2 Dextran modified by sodium N-methylcarboxylate carbamate and sodium L-phenylalaninate carbamate DP - 19 i = 1.1 J = 0.9 Polymer 12 Dextran modified by sodium N-methylcarboxylate carbamate and sodium L-phenylalaninate carbamate DP = 19 i = 0.4 j = 0.6 Oligomer 13 Dextran modified by sodium N-methylcarboxylate carbamate and sodium L-phenylalaninate carbamate DP = 4 i = 1.5 j = 1.5 | 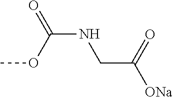 | 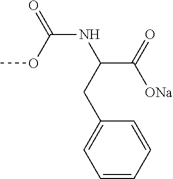 |
| Polymer 3 Dextran modified by sodium N-methytcarboxylate carbamate and octyl phenylalaninate carbamate DP = 19 i = 1.1 j = 0.1 | 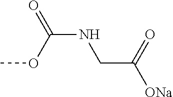 | 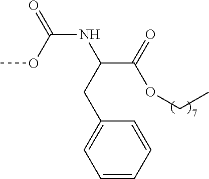 |
| Polymer 4 Dextran modified by sodium L-aspartate acid carbamate and sodium L- phenylalaninate carbamate DP = 19 i = 0.8 j = 1.2 | 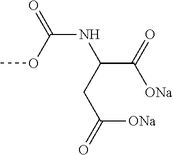 | 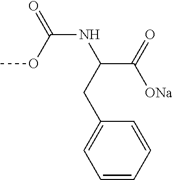 |
| Polymer 5 Dextran modified by sodium 6-hexanoate carbamate and benzyl phenylalaninate carbamate DP = 19 i = 1.05 j = 0.7 | 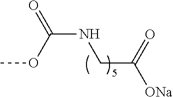 | 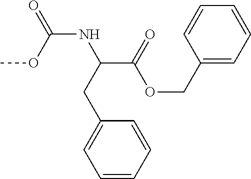 |
| Polymer 6 Dextran modified by sodium 6-hexanoate carbamate and sodium L-leucinate carbamate DP = 19 i = 1.2 j = 0.5 | 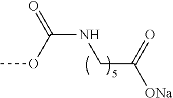 | 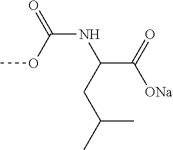 |
| Polymer 7 Dextran modified by sodium L-phenylalaninate L-aspartate carbamate DP = 19 i = 1 j = 0 | 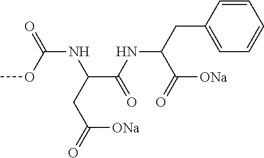 | N/A |
| Polymer 8 Dextran modified by sodium 2-[(2-aminoethoxy- carbonyl)amino]-3- phenylpropanoate carbamate DP = 19 i = 1 j = 0 | 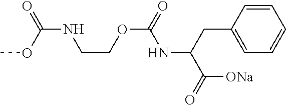 | N/A |
| Polymer 9 Dextran modified by sodium N-methylcarboxylate carbamate and cholesteryl leucinate carbamate DP = 19 i = 1.96 j = 0.05 | 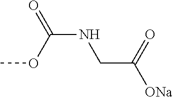 | 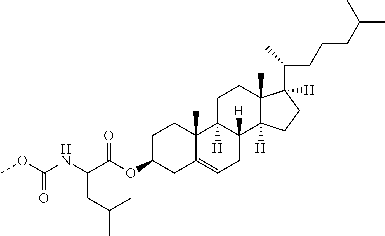 |
| Polymer 10 Dextran modified by sodium N-methylcarboxylate carbamate and cholesteryl 1-ethylenediamine- carboxylate carbamate DP = 19 i = 1.96 j = 0.05 | 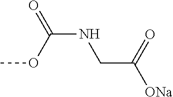 | 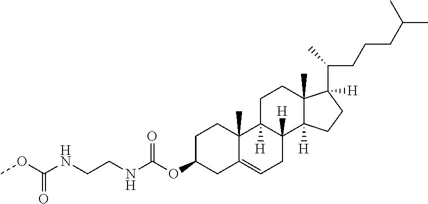 |

### Example 1

### Dextran Modified by Sodium L-Phenylalaninate Carbamate

Polymer 1

Ethyl L-phenylalaninate isocyanate is obtained according to the process described in the publication (Tsai, J. H. et al., Organic Syntheses, 2004, 10, 544-545) from ethyl L-phenylalanine hydrochloride (Bachem) and triphosgene (Sigma).

The grafting of ethyl L-phenylalaninate isocyanate to dextran is carried out according to the process described in the publication (Arranz, F. et al., Makromol. Chemie, 1987, 188, 2831-2838). 4 g (i.e., 0.07 mol of hydroxyl functional groups) of dextran with a weight-average molar mass of approximately 5 kg/mol (Pharmacosmos) are dissolved in a DMF/DMSO mixture in the presence of DABCO (1,4-diazabicyclo[2.2.2]octane). Toluene is added to the mixture and the medium is dehydrated by heteroazeotropic distillation. 8.1 g (0.04 mol) of ethyl L-phenylalaninate isocyanate are gradually introduced at 80° C. After reacting for 12 hours, the medium is diluted in water and purified by diafiltration over a 5 kD PES membrane against 0.1N NaOH, 0.9% NaCl and water. The final solution is quantitatively determined by solids content, in order to determine the concentration of polymer, and then quantitatively determined by acid/base titration in water/acetone 50/50 (v/v), in order to determine the degree of functionalization of the hydroxyls to give sodium L-phenylalaninate carbamate.

According to the solids content: [Polymer 1]=16.1 mg/g

According to the acid/base titration: the degree of functionalization of the hydroxyls to give sodium L-phenylalaninate carbamate is 0.9.

### Example 2

### Dextran Modified by Sodium N-Methylcarboxylate Carbamate and Sodium L-Phenylalaninate Carbamate

Polymer 2

Ethyl L-phenylalaninate isocyanate is obtained according to the process described in the publication (Tsai, J. H. et al., Organic Syntheses, 2004, 10, 544-545) from ethyl L-phenylalanine hydrochloride (Bachem) and triphosgene (Sigma).

Ethyl isocyanatoacetate is available from Aldrich.

By a process similar to Example 1, starting from a dextran with a weight-average molar mass of approximately 5 kg/mol (Pharmacosmos), a dextran modified by sodium N-methylcarboxylate carbamate and sodium L-phenylalaninate carbamate is obtained.

According to the solids content: [Polymer 2]=16.7 mg/g

According to the acid/base titration and the ¹H NMR: the degree of functionalization of the hydroxyls to give sodium N-methylcarboxylate carbamate is 1.1 and the degree of functionalization of the hydroxyls to give sodium L-phenylalaninate carbamate is 0.8.

### Example 3

### Dextran Modified by Sodium N-Methylcarboxylate Carbamate and Octyl Phenylalaninate Carbamate

Polymer 3

By a process similar to Example 1, starting from a dextran with a weight-average molar mass of approximately 40 kg/mol (Pharmacosmos), a dextran modified by sodium N-methylcarboxylate carbamate is obtained.

According to the solids content: [polymer]=19.5 mg/g

According to the acid/base titration: the degree of functionalization of the hydroxyl functional groups to give sodium N-methylcarboxylate carbamate is 1.1.

The solution of dextran modified by sodium N-methylcarboxylate carbamate is passed over a Purolite resin (anionic) in order to obtain the dextran modified by N-methylcarboxylic acid carbamate, which is subsequently lyophilized for 18 hours.

Octyl phenylalaninate, para-toluenesulphonic acid salt, is obtained according to the process described in the patent (Kenji, M. et al., U.S. Pat. No. 4,826,818).

Octyl phenylalaninate isocyanate is obtained according to the process described in the publication (Tsai, J. H. et al., Organic Syntheses, 2004, 10, 544-545) from octyl phenylalaninate, para-toluenesulphonic acid salt, and triphosgene (Sigma).

By a process similar to Example 1, without the use of 0.1N sodium hydroxide solution during the diafiltration stage, starting from dextran modified by N-methylcarboxylic acid carbamate, a dextran modified by sodium N-methylcarboxylate carbamate and octyl phenylalaninate carbamate is obtained.

According to the solids content: [Polymer 3]=17.5 mg/g

According to the acid/base titration and the ¹H NMR: the degree of functionalization of the hydroxyl functional groups to give sodium N-methylcarboxylate carbamate is 1.1 and the degree of functionalization of the hydroxyl functional groups to give octyl phenylalaninate carbamate is 0.1.

### Example 4

### Dextran Modified by Sodium L-Aspartate Acid Carbamate and Sodium L-Phenylalaninate Carbamate

Polymer 4

Ethyl L-phenylalaninate isocyanate is obtained according to the process described in the publication (Tsai, J. H. et al., Organic Syntheses, 2004, 10, 544-545) from ethyl L-phenylalanine hydrochloride (Bachem) and triphosgene (Sigma).

Dimethyl L-aspartate isocyanate is obtained according to the process described in the publication (Tsai, J. H., et al., Organic Syntheses, 2004, 10, 544-545) from dimethyl L-aspartate hydrochloride (Bachem) and triphosgene (Sigma).

By a process similar to Example 1, starting from a dextran with a weight-average molar mass of approximately 5 kg/mol (Pharmacosmos), a dextran modified by sodium L-aspartate carbamate and sodium L-phenylalaninate carbamate is obtained.

According to the solids content: [Polymer 4]=21 mg/g

According to the acid/base titration and the ¹H NMR: the degree of functionalization of the hydroxyl functional groups to give sodium L-aspartate carbamate is 0.8 and the degree of functionalization of the hydroxyl functional groups to give sodium L-phenylalaninate carbamate is 1.2.

### Example 5

### Dextran Modified by Sodium 6-Hexanoate Carbamate and Benzyl Phenylalaninate Carbamate

Polymer 5

Methyl 6-aminohexanoate isocyanate is obtained according to the process described in the publication (Tsai, J. H. et al., Organic Syntheses, 2004, 10, 544-545) from methyl 6-aminohexanoate hydrochloride (Aldrich) and triphosgene (Sigma).

By a process similar to Example 1, starting from a dextran with a weight-average molar mass of approximately 10 kg/mol (Pharmacosmos), a dextran modified by sodium 6-hexanoate carbamate is obtained.

According to the solids content: [polymer]=23.5 mg/g

According to the acid/base titration: the degree of functionalization of the hydroxyl functional groups to give sodium 6-hexanoate carbamate is 1.05.

The solution of dextran modified by sodium 6-hexanoate carbamate is passed over a Purolite resin (anionic) in order to obtain a dextran modified by 6-hexanoic acid carbamate which is subsequently lyophilized for 18 hours.

Benzyl L-phenylalaninate isocyanate is obtained according to the process described in the publication (Tsai, J. H. et al., Organic Syntheses, 2004, 10, 544-545) from benzyl L-phenylalaninate hydrochloride (Bachem) and triphosgene (Sigma).

By a process similar to Example 1, without the use of 0.1N sodium hydroxide solution during the diafiltration stage, starting from dextran modified by 6-hexanoic acid carbamate, a dextran modified by sodium 6-hexanoate carbamate and benzyl L-phenylalaninate carbamate is obtained.

According to the solids content: [Polymer 5]=19.6 mg/g

According to the acid/base titration and the ¹H NMR: the degree of functionalization of the hydroxyl functional groups to give sodium 6-hexanoate carbamate is 1.05 and the degree of functionalization of the hydroxyl functional groups to give benzyl L-phenylalaninate carbamate is 0.7.

### Example 6

### Dextran Modified by Sodium 6-Hexanoate Carbamate and Sodium L-Leucinate Carbamate

Polymer 6

Methyl 6-aminohexanoate isocyanate is obtained according to the process described in the publication (Tsai, J. H. et al., Organic Syntheses, 2004, 10, 544-545) from methyl 6-aminohexanoate hydrochloride (Aldrich) and triphosgene (Sigma).

Ethyl L-leucinate isocyanate is obtained according to the process described in the publication (Tsai, J. H. et al., Organic Syntheses, 2004, 10, 544-545) from ethyl L-leucinate hydrochloride (Bachem) and triphosgene (Sigma).

By a process similar to Example 1, starting from a dextran with a weight-average molar mass of approximately 5 kg/mol (Pharmacosmos), a dextran modified by sodium 6-hexanoate carbamate and sodium L-leucinate carbamate is obtained.

According to the solids content: [Polymer 6]=22 mg/g

According to the acid/base titration and the ¹H NMR: the degree of functionalization of the hydroxyls to give sodium 6-hexanoate carbamate is 1.2 and the degree of functionalization of the hydroxyls to give sodium L-leucinate carbamate is 0.5.

### Example 7

### Dextran Modified by Sodium L-Phenylalaninate L-Aspartate Carbamate

Polymer 7

Ethyl L-phenylalaninate β-benzyl-L-aspartate hydrochloride is synthesized according to a peptide coupling process described in the publication (Carpino et al., J. Org. Chem., 1995, 60, 3561) from 2-[(tert-butoxycarbonyl)amino]-4-benzyloxy-4-oxobutanoic acid (Boc-Asp(OBzl)-OH, available from Bachem) and ethyl L-phenylalaninate hydrochloride, HCl-PheOEt, followed by deprotection of the tert-butoxycarbonyl (Boc) group in hydrochloric acid at 0° C.

The isocyanate of the dipeptide ethyl L-phenylalaninate β-benzyl-L-aspartate is obtained according to the process described in the publication (Tsai, J. H. et al., Organic Syntheses, 2004, 10, 544-545) from ethyl L-phenylalaninate β-benzyl-L-aspartate hydrochloride and triphosgene (Sigma).

By a process similar to Example 1, starting from a dextran with a weight-average molar mass of approximately 5 kg/mol (Pharmacosmos), a dextran modified by sodium L-phenylalaninate L-aspartate dipeptide carbamate is obtained.

According to the solids content: [Polymer 7]=21.0 mg/g

According to the ¹H NMR: the degree of functionalization of the hydroxyls to give sodium L-phenylalaninate L-aspartate carbamate is 1.0.

### Example 8

### Dextran Modified by Sodium 2-[(2-aminoethoxycarbonyl)amino]-3-phenylpropanoate carbamate

Polymer 8

Ethyl 2-[(2-aminoethoxycarbonyl)amino]-3-phenylpropanoate hydrochloride is synthesized according to the coupling process described in the publication (Ouari, O. et al., J. Org. Chem., 1999, 64, 3554-3556) from tert-butyl 2-hydroxyethylcarbamate and ethyl L-phenylalaninate isocyanate, followed by deprotection of the tert-butoxycarbonyl (Boc) group in hydrochloric acid at 0° C.

Ethyl 2-[(2-aminoethoxycarbonyl)amino]-3-phenylpropanoate isocyanate is obtained according to the process described in the publication (Tsai, J. H. et al., Organic Syntheses, 2004, 10, 544-545) from ethyl 2-[(2-aminoethoxycarbonyl)amino]-3-phenylpropanoate hydrochloride and triphosgene (Sigma).

By a process similar to Example 1, starting from a dextran with a weight-average molar mass of approximately 5 kg/mol (Pharmacosmos), a dextran modified by sodium 2-[(2-aminoethoxycarbonyl)amino]-3-phenylpropanoate carbamate is obtained.

According to the solids content: [Polymer 8]=18.0 mg/g

According to the ¹H NMR: the degree of functionalization of the hydroxyls to give sodium 2-[(2-aminoethoxycarbonyl)amino]-3-phenylpropanoate carbamate is 1.0.

### Example 9

### Dextran Modified by Sodium N-Methylcarboxylate Carbamate and Cholesteryl Leucinate Carbamate

Polymer 9

By a process similar to Example 1, starting from a dextran with a weight-average molar mass of approximately 5 kg/mol (Pharmacosmos), a dextran modified by sodium N-methylcarboxylate carbamate is obtained.

According to the solids content: [polymer]=24.0 mg/g

According to the acid/base titration: the degree of functionalization of the hydroxyl functional groups to give sodium N-methylcarboxylate carbamate is 2.0.

The solution of dextran modified by sodium N-methylcarboxylate carbamate is passed over a Purolite resin (anionic) in order to obtain the dextran modified by N-methylcarboxylic acid carbamate which is subsequently lyophilized for 18 hours.

Cholesteryl leucinate, para-toluenesulphonic acid salt, is obtained according to the process described in the patent (Kenji, M. et al., U.S. Pat. No. 4,826,818).

Cholesteryl leucinate isocyanate is obtained according to the process described in the publication (Tsai, J. H. et al., Organic Syntheses, 2004, 10, 544-545) from cholesteryl leucinate, para-toluenesulphonic acid salt, and triphosgene (Sigma).

By a process similar to Example 1, without the use of 0.1N sodium hydroxide solution during the diafiltration stage, starting from dextran modified by N-methylcarboxylic acid carbamate, a dextran modified by sodium N-methylcarboxylate carbamate and cholesteryl leucinate carbamate is obtained.

According to the solids content: [Polymer 9]=16.1 mg/g

According to the ¹H NMR: the degree of functionalization of the hydroxyl functional groups to give sodium N-methylcarboxylate carbamate is 2.0 and the degree of functionalization of the hydroxyl functional groups to give cholesteryl leucinate carbamate is 0.05.

### Example 10

### Dextran Modified by Sodium N-Methylcarboxylate Carbamate and Cholesteryl 1-Ethylenediaminecarboxylate Carbamate

Polymer 10

Cholesteryl 1-ethylenediaminecarboxylate hydrochloride is obtained according to the process described in the patent (Akiyoshi, K. et al., WO2010053140).

Cholesteryl 1-ethylenediaminecarboxylate isocyanate is obtained according to the process described in the publication (Tsai, J. H. et al., Organic Syntheses, 2004, 10, 544-545) from cholesteryl 1-ethylenediaminecarboxylate hydrochloride and triphosgene (Sigma).

By a process similar to Example 1, starting from a dextran with a weight-average molar mass of approximately 5 kg/mol (Pharmacosmos), a dextran modified by sodium N-methylcarboxylate carbamate and cholesteryl 1-ethylenediamine-carboxylate carbamate is obtained.

According to the solids content: [Polymer 10]=19.7 mg/g

According to the ¹H NMR: the degree of functionalization of the hydroxyls to give sodium N-methylcarboxylate carbamate is 2.0 and the degree of functionalization of the hydroxyls to give cholesteryl 1-ethylenediaminecarboxylate carbamate is 0.05.

### Example 11

### Dextran Modified by Sodium L-Phenylalaninate Carbamate

Polymer 11

By a process similar to Example 1, starting from a dextran with a weight-average molar mass of approximately 5 kg/mol (Pharmacosmos), a dextran modified by sodium L-phenylalaninate carbamate is obtained.

According to the solids content: [Polymer 11]=21.3 mg/g

According to the acid/base titration: the degree of functionalization of the hydroxyls to give sodium L-phenylalaninate carbamate is 2.0.

### Example 12

### Dextran Modified by Sodium N-Methylcarboxylate Carbamate and Sodium L-Phenylalaninate Carbamate

Polymer 12

By a process similar to Example 1, starting from a dextran with a weight-average molar mass of approximately 5 kg/mol (Pharmacosmos), a dextran modified by sodium N-methylcarboxylate carbamate and sodium L-phenylalaninate carbamate is obtained.

According to the solids content: [Polymer 12]=23.1 mg/g

According to the acid/base titration and the ¹H NMR: the degree of functionalization of the hydroxyls to give sodium N-methylcarboxylate carbamate is 0.4 and the degree of functionalization of hydroxyls to give sodium L-phenylalaninate carbamate is 0.6.

### Example 13

### Dextran Modified by Sodium N-Methylcarboxylate Carbamate and Sodium L-Phenylalaninate Carbamate

Oligomer 13

By a process similar to Example 1, starting from a dextran with a weight-average molar mass of approximately 1 kg/mol (Pharmacosmos), a dextran modified by sodium N-methylcarboxylate carbamate and sodium L-phenylalaninate carbamate is obtained.

According to the solids content: [Oligomer 13]=11.7 mg/g

According to the acid/base titration and the ¹H NMR: the degree of substitution to give sodium N-methylcarboxylate carbamate is 1.5 and the degree of substitution to give sodium L-phenylalaninate carbamate is 1.5.

### Example 14

### Dextran Modified by Sodium L-Phenylalaninate Carbamate

Oligomer 14

By a process similar to Example 1, starting from a dextran with a weight-average molar mass of approximately 1 kg/mol (Pharmacosmos), a dextran modified by sodium N-methylcarboxylate carbamate and sodium L-phenylalaninate carbamate is obtained.

According to the solids content: [Oligomer 14]=19.9 mg/g

According to the acid/base titration and the ¹H NMR: the degree of substitution to give sodium L-phenylalaninate carbamate is 2.1.

### Example 15

### Decomposition of the Polymers In Vitro

Several tests can be carried out in order to study the ability of the polymers to decompose in vitro, such as those described in the review by C. Larsen (Advanced Drug Delivery Reviews, 3 (1989) 103-154). Some of these tests, in particular at acidic and basic pH, have made it possible to demonstrate that the polymers according to the invention exhibit a decomposition due to the hydrolysis of the grafts of the polysaccharide which is favourable from the viewpoint of the therapeutic application.

1. Anionic polysaccharide, wherein it is chosen from polysaccharides comprising carboxylic acid functional groups of formula I: 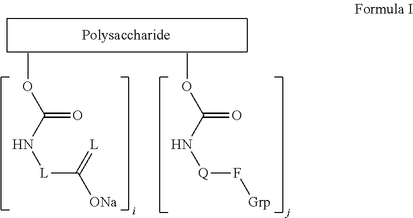
in which:
the said polysaccharide is chosen from the group consisting of dextran, alginate, hyaluronan and galacturonan, these being natural, synthetic or modified,
i represents the degree of carboxylate functionalization of the hydroxyls carried per saccharide unit and 0<i≦3, and
j represents the degree of functionalization of the hydroxyls carried per saccharide unit and 0≦j≦1.5 with 0<i+j≦3, and
if j=0, then L is not —CH₂—,
L and Q, which are different, represent an optionally branched and/or unsaturated chain comprising from 1 to 15 carbons, optionally comprising one or more heteroatoms, such as O, N and/or S, and optionally comprising one or more saturated, unsaturated or aromatic rings or heterocycles, and are chosen from the radicals of Formulae VII and VIII: 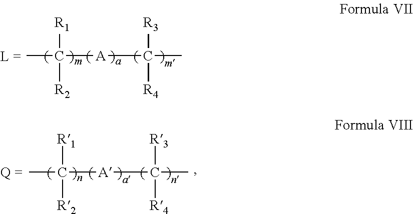
in which:
m and n, which are identical or different, are greater than or equal to 1 and less than or equal to 12,
m′ and n′, which are identical or different, are less than or equal to 12,
A and A′, which are identical or different, are functional groups chosen from the group consisting of the amide functional group, the carbonyl being bonded to the —Cₘ(R₁R₂)— radical, such as —C(R₁R₂)—CO—NH—C(R₃R₄)—, or bonded to the —Cₙ(R′₁R′₂)— radical, such as —C(R′₁R′₂)—CO—NH—C(R′₃R′₄)—, and the carbamate functional group,
R₁ and R₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₁ and R′₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R₃ and R₄, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₃ and R′₄, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
a and a′, which are identical or different, are equal to 0 or 1,
when R₁ and R₂ are hydrogen atoms and when a=0 and m′=0, then m is not 1,
when a′=1, then L is not: 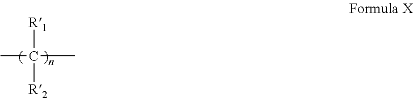
in which R′₁, R′₂ and n have the values defined above,
F is a functional group chosen from the group consisting of the carbamate functional group, the ester functional group, the carbonyl being bonded to the Q radical, such as -Q-CO—O-Grp, the amide functional group, the carbonyl being bonded to the Q radical, such as -Q-CO—NH-Grp, and the carboxylate functional group, the carbonyl being bonded to the Q radical, such as -Q-CO—OH,
Grp is chosen from the group consisting of a mono- or divalent cation, if the —NH-Q-F— radical results from an amino acid, F being a carboxylate functional group, and of a chain comprising from 4 to 50 carbons, which is optionally branched and/or unsaturated, and/or comprising one or more heteroatoms, such as O, N and/or S, in the form of non-terminal functional groups, and/or comprising one or more saturated, unsaturated or aromatic rings or heterocycles, and
the carboxylic acid functional groups are in the form of carboxylates of mono- or divalent cations.

2. Anionic polysaccharide according to claim 1, wherein it is chosen from the polysaccharides of formula I, in which:
j, L and Q have the meanings given above,
F is a functional group chosen from the group consisting of the carbamate functional group, the ester functional group, the carbonyl being bonded to the Q radical, such as -Q-CO—O-Grp, and the amide functional group, the carbonyl being bonded to the Q radical, such as -Q-CO—NH-Grp,
Grp is chosen from the group consisting of a chain comprising from 4 to 50 carbons, which is optionally branched and/or unsaturated, and/or comprising one or more heteroatoms, such as O, N and/or S, in the form of non-terminal functional groups, and/or comprising one or more saturated, unsaturated or aromatic rings or heterocycles,
the carboxylic acid functional groups are in the form of carboxylates of mono- or divalent cations.

3. Anionic polysaccharide according to claim 1, wherein it is chosen from the polysaccharides of formula I, in which:
i, j, F, L and Q have the meanings given above,
Grp is chosen from the group consisting of a mono- or divalent cation, if the —NH-Q-F— radical results from an amino acid, F being a carboxylate functional group, a saturated or unsaturated and linear, branched or cyclic C₄ to C₁₆ alkyl, a benzyl, an alkylaryl and a cholesteryl,
the carboxylic acid functional groups are in the form of carboxylates of mono- or divalent cations.

4. Anionic polysaccharide according to claim 1, wherein it is chosen from the polysaccharides of formula IX: 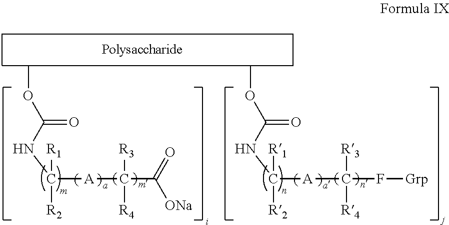
in which:
i, j, m, m′, a, a′, n and n′ have the meanings given in claim 1,
A and A′, which are identical or different, are functional groups chosen from the group consisting of the amide functional group, the carbonyl being bonded to the —Cₘ(R₁R₂)— radical, such as —C(R₁R₂)—CO—NH—C(R₃R₄)—, or bonded to the —Cₙ(R′₁R′₂)— radical, such as —C(R′₁R′₂)—CO—NH—C(R′₃R′₄)—, and the carbamate functional group,
R₁ and R₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₁ and R′₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R₃ and R₄, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₃ and R′₄, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
F is a functional group chosen from the group consisting of the carbamate functional group, the ester functional group, the carbonyl being bonded to the Q radical, such as -Q-CO—O-Grp, the amide functional group, the carbonyl being bonded to the Q radical, such as -Q-CO—NH-Grp, and the carboxylate functional group, the carbonyl being bonded to the Q radical, such as -Q-CO—OH,
Grp is chosen from the group consisting of a mono- or divalent cation, if the —NH-Q-F-radical results from an amino acid, F being a carboxylate functional group, a saturated or unsaturated and linear, branched or cyclic C₁ to C₆ alkyl, a benzyl, an alkylaryl and a cholesteryl, and
the carboxylic acid functional groups are in the form of carboxylates of mono- or divalent cations.

5. Anionic polysaccharide according to claim 4, wherein it is chosen from the polysaccharides of formula I, in which:
i, j, F and Grp have the meanings given in claim 1,
a=0, a′=0, m′=0, n′=0
L and Q, which are different, are chosen from the radicals of formulae II and III 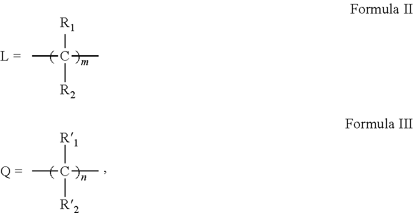
in which:
m and n, which are identical or different, are greater than or equal to 1 and less than or equal to 12, and
if j=0, then m is not 1 when R₁ and R₂ are hydrogen atoms,
R₁ and R₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups,
R′₁ and R′₂, which are identical or different, are chosen from the group consisting of a hydrogen atom, a saturated or unsaturated and linear, branched or cyclic C₁ to C₆ alkyl, a benzyl and an alkylaryl, the said groups optionally comprising heteroatoms chosen from the group consisting of O, N and S, in the form of functional groups chosen from the group consisting of the carboxylic acid —C(O)OH, alcohol —OH, phenol —C₆H₄—OH and thiol —SH functional groups.

6. Anionic polysaccharide according to claim 1, wherein it is chosen from the polysaccharides of formula IX, in which:
j, F, L and Q have the meanings given above, and
Grp is chosen from the group consisting of a mono- or divalent cation, if the —NH-Q-F— radical results from an amino acid and F is a carboxylate functional group, and of a chain comprising from 4 to 50 carbons, which is optionally branched and/or unsaturated, and/or comprising one or more heteroatoms, such as O, N and/or S, in the form of non-terminal functional groups, and/or comprising one or more saturated, unsaturated or aromatic rings or heterocycles.

7. Anionic polysaccharide according to claim 1, wherein it is chosen from the polysaccharides of formula IV: 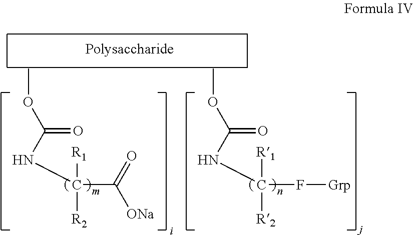
in which:
i, j, m and n have the meanings given above,
R₁ and R₂, which are identical or different, are chosen from the group consisting of a hydrogen atom, a saturated or unsaturated and linear, branched or cyclic C₁ to C₆ alkyl, a benzyl and an alkylaryl, the said groups optionally comprising carboxylic acid functional groups,
R′₁ and R′₂, which are identical or different, are chosen from the group consisting of a hydrogen atom, a saturated or unsaturated and linear, branched or cyclic C₁ to C₆ alkyl, a benzyl and an alkylaryl,
F is a functional group chosen from the group consisting of the carbamate functional group, the ester functional group, the carbonyl being bonded to the Q radical, such as -Q-CO—O-Grp, the amide functional group, the carbonyl being bonded to the Q radical, such as -Q-CO—NH-Grp, and the carboxylate functional group, the carbonyl being bonded to the Q radical, such as -Q-CO—OH,
Grp is chosen from the group consisting of a mono- or divalent cation, if the —NH-Q-F-radical results from an amino acid and F is a carboxylate functional group, a saturated or unsaturated and linear, branched or cyclic C₁ to C₆ alkyl, a benzyl, an alkylaryl and a cholesteryl, and
the carboxylic acid functional groups are in the form of a salt.

8. Polysaccharide according to claim 1, wherein the L radical is chosen from the group consisting of the radicals of formula —[CH₂]ₒ— with 1≦o≦6.

9. Polysaccharide according to claim 1, wherein the L radical is chosen from the group consisting of the following radicals:

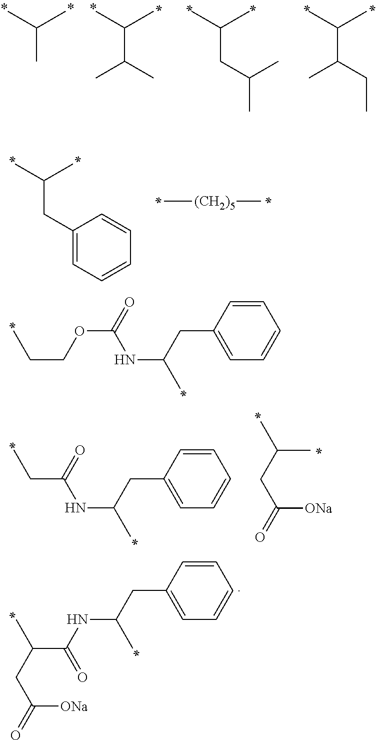

10. Polysaccharide according to claim 1, wherein the Q radical is chosen from the group consisting of the following radicals:

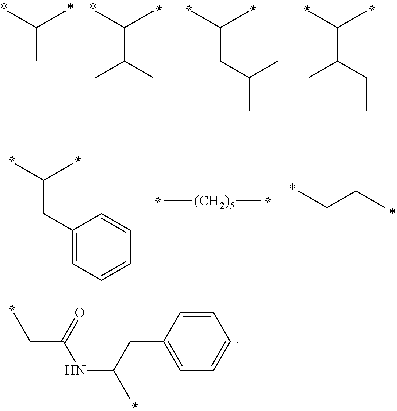

11. Polysaccharide according to claim 1 wherein the Grp radical is chosen from the group consisting of the radicals of formula:
—[CH₂]ₚ—CH₃ with 4≦p≦12.

12. Polysaccharide according to claim 1, wherein the -Grp radical is a benzyl radical.

13. Polysaccharide according to claim 1, wherein the polysaccharide is a natural polysaccharide and is chosen from the group consisting of dextran, alginate, hyaluronan and galacturonan.

14. Polysaccharide according to claim 1, wherein the polysaccharide is a natural polysaccharide carrying carboxylic acid functional groups and is chosen from the group consisting of alginate, hyaluronan and galacturonan.

15. Polysaccharide according to claim 1, wherein the polysaccharide is a neutral natural polysaccharide and is dextran.

16. Polysaccharide according to claim 1, wherein the polysaccharide is a modified synthetic polysaccharide obtained from a polysaccharide chosen from the group consisting of dextran, alginate, hyaluronan and galacturonan substituted, by etherification of at least one hydroxyl functional group of the polysaccharide, by a radical carrying a carboxylic acid functional group, the said radical being chosen from the group consisting of the radicals of following formula V: 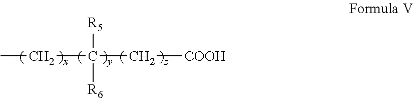
in which:
1≦x+y+z≦6, 0≦x≦3, 0≦y≦3 and 0≦z≦3
R₅ and R₆, which are identical or different, are chosen from the group consisting of —H, linear or branched C₁ to C₃ alkyl, —COOH and the radical of general formula VI: 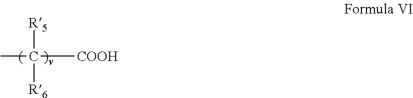
in which:
1≦v≦3 and
R′₅ and R′₆, which are identical or different, are chosen from the group consisting of —H and a linear or branched C₁ to C₃ alkyl group.

17. Polysaccharide according to claim 1, wherein the polysaccharide is a synthetic modified polysaccharide is a carboxymethyldextran.

18. Polysaccharide according to claim 1, wherein the polysaccharide is a polysaccharide chosen from the group consisting of the following polysaccharides:
dextran modified by sodium L-phenylalaninate carbamate,
dextran modified by sodium N-methylcarboxylate carbamate and sodium L-phenylalaninate carbamate,
dextran modified by sodium N-methylcarboxylate carbamate and octyl phenylalaninate carbamate,
dextran modified by sodium L-aspartate carbamate and sodium L-phenylalaninate carbamate,
dextran modified by sodium 6-hexanoate carbamate and benzyl phenylalaninate carbamate,
dextran modified by sodium 6-hexanoate carbamate and sodium L-leucinate carbamate,
dextran modified by sodium L-phenylalaninate L-aspartate carbamate,
dextran modified by sodium 2-[(2-aminoethoxycarbonyl)amino]-3-phenylpropanoate carbamate,
dextran modified by sodium N-methylcarboxylate carbamate and cholesteryl leucinate carbamate,
dextran modified by sodium N-methylcarboxylate carbamate and cholesteryl 1-ethylenediamine carboxylate carbamate.

19. A pharmaceutical composition comprising the anionic polysaccharide according to claim 1.

20. A pharmaceutical composition comprising anionic polysaccharide according to claim 1 and at least one active principle.

21. Pharmaceutical composition according to claim 17, wherein the active principle is chosen from the group consisting of proteins, glycoproteins, peptides and non-peptide therapeutic molecules.
